(19) United States
(12) Patent Application Publication
(10) Pub. No.: US 2009/0149941 A1
(43) Pub. Date: Jun. 11, 2009

(54) Compressible tubular tissue supports

(76) Inventors: Ali Abdullah Tareq Hasson, Aachen (DE); Dagmar Boltersdorf, Kreuzau (DE); Ralf Werner Schmidt, Aachen (DE)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 12/095,266

(22) PCT Filed: Nov. 27, 2006
(86) PCT No.: PCT/IB2006/004248

§ 371 Date: Nov. 17, 2008

(30) Foreign Application Priority Data
Nov. 28, 2005 (DE) 10 2005 056 529.8

(51) Int. Cl. A61F 2/06

(52) U.S. Cl. 623/1.15; 623/1.17; 623/1.2

(57) ABSTRACT

The invention provides a novel compressed tubular tissue supports (1) that can easily be introduced into vessels requiring support.

### CROSS REFERENCE TO RELATED APPLICATIONS

This application claims priority under 35 U.S.C. § 119 to German Application No. 10 2005 056 529.8, filed Nov. 28, 2005, which application is expressly incorporated herein by reference in its entirety.

### BACKGROUND OF THE INVENTION

1. Field of the Invention

The invention relates to compressible tubular tissue supports (stents), a process for their production and use thereof.

2. Description of the Related Art

A stent (medical technology) is an implant which is introduced into hollow organs (e.g., veins or arteries, bile ducts, the trachea, the oesophagus) in order to brace the wall radially outwards. Stents are used, for example, in coronary vessels for prophylaxis of restenosis after PTCA (percutaneous transluminal coronary angioplasty).

Stents are small grid structures in the form of a tube composed of metal or of polymers, often used in the context of angioplasty, in which strictures in vessels are widened. In cancer treatment, stents serve to prevent closure of strictures caused by malignant tumours in respiratory passages, bile ducts or the oesophagus, after these have been expanded.

Stents are usually cylindrical products composed of a type of wire mesh (wire coil design) or of tubes, which may be perforated or unperforated (slotted tube design). The length of commonly used stents range from 1 to 12 cm, and their diameter ranges from 1 to 12 mm.

A stent is subject to various requirements. First, the support has to exert large radial forces on the hollow organ requiring support. Second, the stent must be able to be compressed radially to permit its easy introduction into a hollow organ without at the same time injuring the vessel wall or the surrounding tissue. The solution to this problem involves using the stents in compressed form and not expanding them until they are placed in the correct location. In the compressed condition, the diameter is markedly smaller than in the expanded condition.

Two different technologies are used (see, e.g., Market report entitled “US Peripheral and Vascular Stent and AAA Stent Graft Market,” Frost & Sullivan, (2001)) for minimally invasive stent use: (1) expandable-balloon stents (system composed of balloon, catheter, stent); and (2) self-expandable stents (system composed of introductory sheath (protective sheath), catheter, stent).

Self-expanding stents are generally composed of shape-memory materials (SM materials). Shape-memory materials are materials which change their external shape on exposure to an external stimulus. The materials are by way of example capable of controlled change in their shape when the temperature is increased above what is known as the switching temperature (Tₜᵣₐₙₛ). The shape-memory effect is utilized for “spontaneous” enlargement of the diameter of the stent, and to fix the stent at the location of use. The shape-memory effect is not a specific property of any of the materials. Rather, it is a direct result of the combination of structure and morphology and of a processing/programming technology.

In shape-memory materials, a distinction is made between a permanent and a temporary shape. The material is first converted to its permanent shape, using conventional processing methods (e.g., extrusion). The material is then converted, reshaped and fixed into its desired temporary shape. This procedure is also termed programming. The conversion process can include either of heating of the specimen, reshaping and a cooling procedure, or else of shaping at relatively low temperature. By these steps, the permanent shape has been held in memory, while the temporary shape is actually present. Heating of the material to a temperature higher than the transition temperature for a change of morphology (switching temperature) triggers the shape-memory effect and thus causes resumption of the permanent shape held in memory.

The shape-memory effect, which permits controlled alteration in the shape of a material by application of an external stimulus is described by way of example in Angew. Chem., 114, 2138-62 (2002).

Introduction of a stent into a hollow organ is difficult When the stent is introduced into the hollow organ there is a risk that the surrounding tissue will be injured by abrasion in the process, because the stent is too large and has sharp edges. The shape-memory effect is therefore also used again to reduce the diameter of the stent when the stent is in turn to be removed. Examples of removable stents composed of metals with shape-memory properties are known, for example, in: U.S. Pat. Nos. 6,413,273; 6,348,067; 5,037,427; and 5,197,978.

Examples of metallic SM materials used are nitinol, an equiatomic alloy composed of nickel and titanium (see, e.g., J. Appl. Phys., 34, 1475 (1963)). However, nitinol cannot be used when a nickel allergy is present. The material is moreover very expensive and programmable only by complicated methods. This programming process needs comparatively high temperatures. Programming in the body is therefore impossible. The SM material is therefore programmed outside the body, i.e. converted to its temporary shape. After implantation, the shape-memory effect is then triggered and the stent is expanded (i.e., regains its permanent shape). Removal of the stent by again utilizing the shape-memory effect is then impossible. Another frequent problem with metallic stents, not only in the vascular sector, is occurrence of restenosis.

In contrast, other metallic stents composed of SM materials, for example those described in U.S. Pat. No. 5,197,978, also permit utilization of the shape-memory effect for stent removal. However, production of these metallic materials is very complicated and tissue compatibility is not always ensured. Inflammation and pain patterns occur because of the poor matching of the mechanical properties of the stent.

The temporary stent described in U.S. Pat. No. 5,716,410 is a spiral composed of a polymeric shape-memory material (SMP). The SMP material comprises an embedded heating wire. The heating wire has connection by way of a catheter shaft to an electrical control unit, the end of the shaft taking the form of a hollow tube pushed over one end of the spiral.

An alternative embodiment of a temporary stent described in DE 10357747, wherein the material of the stent is biodegradable and thereby gradually resolves at the location of use.

U.S. Pat. No. 5,964,744 describes implants, such as tubes and catheters, for the urogenital sector or gastro-intestinal tract composed of polymeric shape-memory materials which include a hydrophilic polymer. In an aqueous medium the material absorbs moisture and thus softens and changes its shape. The material can also soften on heating. In the case of the ureteral stent, the effect is utilized in order to flex the straight ends of the stent at the location of use (e.g., kidneys and bladder). The result is to fix the ureteral stent at the location of use, so that the stent cannot slip during peristaltic movements of the tissue.

WO 2002/041929 describes tubular vessel implants with shape memory which are also suitable, for example, as bile duct stents. The material is an aliphatic, polycarbonate-based thermoplastic polyurethane with biostability.

DE 10226734 describes stent sleeves to be used in combination with stents, wherein the sleeves are manufactured from a memory material that may be plastic or metal and the stent is made out of conventional material. The sleeve may be folded in a specific manner and after its expansion the sleeve is supported by the stent.

WO 2005/044330 describes folded stents made of biocompatible metal or plastic, in particular of gold. Shape memory polymers are not described therein.

WO 2004/010901 describes vascular stents having an elongated sleeve formed of a shape memory polymeric material. In a first configuration the outer surface of the stent sleeve defines a fold along its length and the stents are axially expanding.

WO 2003/099165 describes a medical device having a tubular portion wherein the tubular portion contains two or more slots separated by ribs. Preferably a slot in one row is contiguous with a rib in another row, so that the tube can be folded by inserting ribs in one row into slots in another row. The manufacturing of such tubular devices is difficult, and their perforation pattern is limited, so that it cannot be optimized to the therapeutic needs. Tubes without perforation are not compatible with this technique.

U.S. Pat. No. 6,245,103 describes bioabsorbable, self-expanding stents composed of braided filaments. The stent is compressed by applying an external radial force. The stent has been mounted on a catheter and is held in stressed, compressed condition by an outer sheath. When the stent is expelled from this arrangement its diameter spontaneously enlarges because of the resilience of the elastic material. This change is not the shape-memory effect, which is triggered by an external stimulus (e.g., a temperature increase). Expandable elastic stents, that are temporarily held in the compressed form by a covering are also described in U.S. Pat. No. 6,475,234.

A disadvantage of known stents composed of SM materials is that an alteration in length always takes place during the transition from the permanent to the temporary shape and then again to the permanent shape. Consequently, the placement precision of the stents and their fitting are unsatisfactory.

### SUMMARY OF THE INVENTION

It is an object of the invention to provide novel stents in which, during the transition from the permanent to the temporary shape and then again into the permanent shape, substantially only radial compression and expansion occurs, and length remains approximately (axially) constant to help ensure high placement precision and easy fitting.

In addition, the stent of the invention should be easy to manufacture, exert high radial forces, and it should be possible to produce it as a tube with an arbitrary perforation pattern in the wall to optimally fit it to the therapeutic needs and even without a perforation.

Radially expandable tubular tissue supports based on shape-memory materials have been invented and are characterized in that the tube in the temporary shape of the shape-memory materials has been folded one or more times in its longitudinal axis or has been crimped accordingly. The compressed temporary shape of the inventive stent can be maintained without auxiliary means (e.g., sleeves).

The invented stents substantially do not alter their length during transition from the permanent to the temporary shape and then again to the permanent shape, thus ensuring high placement precision and easy fitting in the hollow organ. In this regard, radial compression is practically all that occurs.

### BRIEF DESCRIPTION OF THE DRAWINGS

The accompanying drawings, which are included to provide further understanding of the invention and are incorporated in and constitute a part of this specification, illustrate embodiments of the invention and together with the description serve to explain the principles of the invention. In the drawings:

FIG. 1 shows a tube of the tissue support in accordance with the invention;

FIG. 2 shows how a fold is produced via impression of a segment; and

FIG. 3 shows the folded stent.

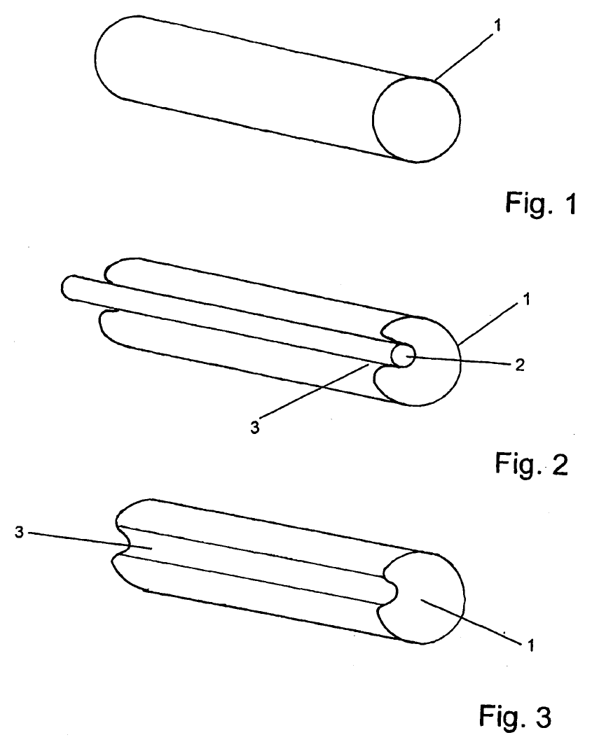

### DETAILED DESCRIPTION OF THE PREFERRED EMBODIMENTS

For the purposes of the invention, stents are generally preferably composed of a polymer in the form of shape-memory material.

For the purposes of the invention, the polymers can include, for example, thermoplastics, blends and networks. Composites composed of biodegradable SMP with inorganic, degradable nanoparticles are also suitable.

The stents of the invention include stents made of an SMP material as well as stents having an underlying structure composed of a biodegradable plastic, embedded or coated with an SMP material. These two substantive designs have a number of advantages.

Stents composed in essence of SMP materials use the SMP material to determine the mechanical properties of the stent. By virtue of the fact that the materials described below are used for this purpose, good tissue compatibility is ensured. Furthermore, as described above, minimally invasive implantation and/or removal of these stents is possible. The SMP materials moreover have relatively good processibility, making the production process easier. Finally, the SMP materials can also be coated or compounded with other substances, thus permitting further functionalization.

If, for example, the underlying structure is composed of a metallic material, then it is preferably composed of biodegradable metals, such as magnesium or magnesium alloys.

The use envisaged for the stent here determines its form based on, for example, the type of surface (microstructuring) or the presence of coatings, etc. For example, the surface of the stent has been formed so as to be compatible with the physiological environment at the location of use, via suitable coating (e.g., hydrogel coating) or surface microstructuring. Parameters such as pH and the number of microbes present have to be considered as a function of the location of use during design of the stent.

Endothelial cells are then used to colonize the surface, and this can be promoted, if appropriate, by suitably modifying the surface (e.g., coating). The result is that growth of endothelial cells gradually covers the stent.

Finally, degradation, usually hydrolytic degradation, begins, and the stent degrades in contact with soft tissue, but, because of the degradation behaviour described above (particle-free degradation, mechanical stability being unimpaired by degradation over a long period), the stent continues to exert the desired supportive action.

In another alternative, the stent is intended to remain outside the endothelial layer after fitting. Such can be achieved by suitable measures, such as selection of the surface, pigment selection for the SMP materials, etc.

Suitable materials for the stents of the present invention are described below.

For the purposes of the invention, SMP materials are materials which by virtue of their chemical and physical structure are capable of carrying out controlled changes of shape. The materials can have, besides their actual permanent shape, another shape which can be impressed temporarily on the material. These materials are characterized by two structural features: crosslinking points (physical or covalent) and switching segments.

SMPs with a thermally induced shape-memory effect have at least one switching segment with a transition temperature in the form of a switching temperature. The switching segments form temporary crosslinking sites which separate on heating above the transition temperature and form again on cooling. The transition temperature can be a glass transition temperature of amorphous regions or a melting point of crystalline regions. The general term “Tₜᵣₐₙₛ” is used below for this temperature.

Above the Tₜᵣₐₙₛ, the material is in the amorphous condition and is elastic. If, therefore, a specimen is heated above the transition temperature Tₜᵣₐₙₛ, then deformed in the flexible condition, and cooled again below the transition temperature, the chain segments are fixed in the deformed condition by virtue of freezing of degrees of freedom (programming). Temporary crosslinking sites (non-covalent) are formed, making it impossible for the specimen to revert to its original shape, regardless of whether any external load is applied. On reheating to a temperature above the transition temperature, these temporary crosslinking sites are again separated and the specimen reverts to its original shape. The temporary shape can be produced again by renewed programming. The precision with which the original shape is regained is termed the recovery ratio.

In photo-switchable SMPs, the function of the switching segment is assumed by photoreactive groups which can be linked to one another reversibly by irradiation with light. In this case, the programming of a temporary shape and regeneration of the permanent shape takes place by virtue of irradiation with no need for any temperature change.

In principle, all SMP materials can be used to produce stents. By way of example, reference may be made here to the materials and the production processes described in DE 10208211 A1, DE 10215858 A1, DE 10217351 A1, DE 10217350 A1, DE 10228120 A1, DE 10253391 A1, DE 10300271 A1, DE 10316573 A1, EP 99934294 A1 and EP 99908402 A1.

SMP materials with two or more temporary shapes have been disclosed in U.S. Pat. No. 6,388,043 (the disclosure of which is expressly incorporated herein by reference in its entirety). When using SMP materials with at least one permanent and two temporary shapes, one of the temporary shapes corresponds to the radially expandable folded shape according to the invention and a second temporary shape to the expanded shape after implantation into the vessel. A further temporary shape or the permanent shape can than be triggered if needed to further expand the stent or to decrease its diameter again. A further expansion can be helpful, if the vessel has a greater inner diameter than was diagnosed or after a restenosis. A triggered contraction can also be useful to better fit the stent to the vessel or to remove the stent. If the stent has more than two temporary forms, expansion as well as contraction can both be triggered at a given time or can be triggered stepwise.

To produce the inventive stents, thermoplastic elastomers can be used. The transition temperature of suitable thermoplastic elastomers is typically approximately 3 to approximately 20° C. above body temperature.

Examples of thermoplastic elastomers are multiblock copolymers. Preferred multiblock copolymers are composed of blocks (macrodiols) composed of α,ω-diol polymers of poly(ε-caprolactone) (PCL), poly(ethylene glycol) (PEG), poly(pentadecalactone), poly(ethylene oxide), poly(propylene oxide), poly(propylene glycol), poly(tetrahydrofuran), poly(dioxanone), poly(lactide), poly(glycolide) and poly(lactide-ran-glycolide) or of α,ω-diol copolymers of the monomers on which the abovementioned compounds are based, in a range of molecular weight (“Mₙ”) of from approximately 250 to approximately 500,000 g/mol. Two different macrodiols are linked with the aid of a suitable bifunctional coupling reagent (specifically an aliphatic or aromatic diisocyanate or diacyl chloride or phosgene) to give a thermoplastic elastomer with Mₙ in the range from approximately 500 to approximately 50,000,000 g/mol. In a phase-segregated polymer, a phase with at least one thermal transition (glass transition or melt transition) can be allocated in each of the blocks of the abovementioned polymer, independently of the other blocks.

Particular preference is given to multiblock copolymers composed of macrodiols based on pentadecalactone (PDL) and ε-caprolactone (PCL) and a diisocyanate. The switching temperature—in this case a melting point—can be adjusted by way of the block length of the PCL in the range from approximately 30 to approximately 55° C. The physical crosslinking points for fixing of the permanent shape of the stent are formed by a second crystalline phase whose melting point is in the range from approximately 87 to approximately 95° C. Blends composed of multiblock copolymers are also suitable. Controlled adjustment of the transition temperatures is possible via the mixing ratio.

In a preferred embodiment, the stents of the invention are made of polymer networks including interpenetrating networks (IPN's). Suitable polymer networks feature covalent crosslinking points and at least one switching segment with at least one transition temperature. The covalent crosslinking points determine the permanent shape of the stent. Suitable IPN's are obtainable by crosslinking of monomers or pre-polymers in the presence of a thermoplastic polymer. Polymer networks and IPN's are particularly applicable in the invention, as folded stents made thereof or at least that include said materials will unfold completely without leaving any kinks. The resulting flat (smooth) surface leads to high stability and superior biocompatibility of the stents.

It has also been found that polymer networks are particularly suitable to incorporate additives like radioactive markers or magnetic particles therein, because the forces exhibited by the shape memory effect and the mechanical stability of the expanded form are only weakened to a minor extend when using polymer networks in combination with such additives. In one embodiment of the invention, the stent includes up to approximately 25 weight-%, preferably approximately 1 to approximately 20 weight-% and particularly preferred approximately 5 to approximately 15 weight-% additives like radioactive markers, or pigments like magnetic particles. The additives are used in an amount as low as possible, that is sufficient for the function of the additive (e.g., for detectability in case of the markers). If magnetic particles are incorporated in the stent of the invention, they are used in an amount to sufficiently heat the stent inductively. As described above, it is preferred that the stent of the invention includes network polymers, if additives in an amount specified above are incorporated therein.

To produce a covalent polymer network, one of the macrodiols described in the above section is crosslinked with the aid of a multifunctional coupling reagent. This coupling reagent can be an at least trifunctional, low-molecular-weight compound, or a polyfunctional polymer. If it is a polymer, it can be a star-shaped polymer with at least three arms, a graft polymer having at least two side chains, a hyperbranched polymer, or a dendritic structure. In the case of the low-molecular-weight, and also the polymeric compounds, the end groups have to be capable of reaction with the diols. Specifically, isocyanate groups can be used for this purpose (polyurethane networks).

Particular preference is given to amorphous polyurethane networks composed of triols and/or tetrols and diisocyanate. Star-shaped prepolymers, such as oligo[(rac-Iactate)co-glycolate]triol or tetrol are prepared via ring-opening copolymerization of rac-dilactide and diglycolide in the melt of the monomers using hydroxy-functional initiators, with addition of dibutyltin(IV) oxide (DBTO) as catalyst. Initiators used for the ring-opening polymerization reaction are ethylene glycol, 1,1,1-tris(hydroxymethyl)ethane and pentaerythritol. Oligo(lactate-co-hydroxycaproate) tetrols and oligooactatehydroxyethoxyacetate)tetrols and [oligo(propylene glycol)-block-oligo (rac-Iactate)-co-glycolate)]triols are produced analogously. The inventive networks can be obtained simply via reaction of the prepolymers with diisocyanate, e.g., with an isomer mixture composed of 2,2,4- and 2,4,4-trimethylhexane 1,6-diisocyanate (TMDI), in solution (e.g., in dichloromethane, and subsequent drying).

The macrodiols described above can moreover be functionalized to give corresponding α,ω-divinyl compounds, which can be crosslinked thermally or photochemically. The functionalization preferably permits covalent linkage of the macromonomers via reactions which give no by-products. This functionalization is preferably rendered available via ethylenically unsaturated units, particularly preferably via acrylate groups and methacrylate groups, particular preference being given to the latter. Specifically, the reaction here to give α,ω-macrodimethacrylates or macrodiacrylates can be carried out via the reaction with the corresponding acyl chlorides in the presence of a suitable base. The networks are obtained via crosslinking of the end-group-functionalized macromonomers. This crosslinking can be achieved by irradiation of the melt, comprising the end-group-functionalized macromonomer component and, if appropriate, a low-molecular-weight comonomer as explained below. Suitable process conditions for crosslinking are irradiation of the mixture in the melt, preferably at temperatures in the range from approximately 40 to approximately 100° C., with light whose wavelength is preferably from approximately 300 to approximately 500 nm. An alternative possibility is thermal crosslinking, if a corresponding initiator system is used.

If the macromonomers described above are crosslinked, the products are networks with a uniform structure if only one type of macromonomer is used. If two types of monomer are used, AB-type networks are obtained. These AB-type networks can also be obtained if the functionalized macromonomers are copolymerized with suitable low-molecular-weight or oligomeric compounds. If the macromonomers have been functionalized with acrylate groups or with methacrylate groups, suitable compounds which can be copolymerized are low-molecular-weight acrylates, methacrylates, diacrylates or dimethacrylates. Preferred compounds of this type are acrylates such as butyl acrylate or hexyl acrylate, and methacrylates such as methyl methacrylate and hydroxyethyl methacrylate.

The amount present of these compounds which can be copolymerized with the macromonomers, based on the network composed of macromonomer and of the low-molecular-weight compound, can be from approximately 5 to approximately 70% by weight, preferably from approximately 15 to approximately 60% by weight. Incorporation of varying amounts of the low-molecular-weight compound takes place via addition of corresponding amounts of compound to the mixture requiring crosslinking. The amount of the low-molecular-weight compound incorporated into the network corresponds to the amount present in the crosslinking mixture.

The macromonomers to be used according to the invention are described in detail below.

Networks with varying crosslinking densities (or segment lengths) and mechanical properties can be achieved by varying the molecular weight of the macrodiols. The number-average molecular weight of the macromonomers requiring covalent crosslinking, determined via GPC analysis, is preferably from approximately 2,000 to approximately 30,000 g/mol, preferably from approximately 5,000 to approximately 20,000 g/mol and particularly preferably from approximately 7,500 to approximately 15,000 g/mol. The macromonomers requiring covalent crosslinking preferably have a methacrylate group at both ends of the macromonomer chain. This type of functionalization permits crosslinking of the macromonomers by simple photoinitiation (irradiation).

The macromonomers are preferably polyester macromonomers, particularly preferably polyester macromonomers based on ε-caprolactone. Other possible polyester macromonomers are based on lactide units, glycolide units, p-dioxanone units and mixtures of these and mixtures with ε-caprolactone units, particular preference being given here to polyester macromonomers having caprolactone units. Other preferred polyester macromonomers are poly(caprolactone-co-glycolide) and poly(caprolactone-co-lactide). The transition temperature can be adjusted by way of the quantitative proportion of the comonomers, as also can the degradation rate.

The macromonomers to be used according to the invention are more preferably polyesters that include the crosslinkable end groups. A particularly preferred polyester to be used according to the invention is a polyester based on ε-caprolactone or pentadecalactone, for which the statements made above concerning molecular weight are applicable. This type of polyester macromonomer, functionalized at the ends, preferably with methacrylate groups, can be prepared via simple syntheses known to the person skilled in the art. These networks, ignoring the other substantive polymeric component of the invention, exhibit semicrystalline properties, and their melting point of the polyester component (which can be determined by DSC measurements) depends on the type of polyester component used and is moreover also controllable thereby. This temperature (Tₘ 1) for segments based on caprolactone units is known to be from approximately 30 to approximately 60° C., depending on the molar mass of the macromonomer.

One preferred network with a melting point as switching temperature is based on the macromonomer poly(caprolactone-co-glycolide) dimethacrylate. The macromonomer can be reacted as it stands, or can be copolymerized with n-butyl acrylate to give the AB network. The permanent shape of the stent is determined by covalent crosslinking points. The network features a crystalline phase whose melting point is capable of controlled adjustment by way of example via the comonomer ratio of caprolactone to glycolide in the range from approximately 20 to approximately 57° C. The function of n-butyl acrylate as comonomer can be, by way of example to optimize the mechanical properties of the stent.

Another preferred network with a glass transition temperature as switching temperature is obtained from an ABA triblock dimethacrylate as macromonomer, characterized by a central block composed of polypropylene oxide and by end blocks A, composed of poly(rac-lactide). The amorphous networks have a very wide switching temperature range.

Examples of particularly preferred polymer networks to be used for the inventive stent are described in the following paragraphs and include semi-crystalline shape memory polymer networks like UV crosslinked dimethacrylate networks, mixed IPN networks and urethane networks. They can be synthesized by methods known in the art. The polymer networks of the invention usually have a gel content of at least approximately 60%, and preferably of at least approximately 70% in the case of mixed IPN networks and even higher gel contents of at least approximately 80% and preferably at least approximately 90% for UV crosslinked dimethacrylate networks and/or urethane networks.

UV crosslinked dimethacrylate networks preferred for the present invention have a high shape recovery of at least approximately 95%, switching temperatures from approximately 40 to 55° C. and show no significant biodegradation within approximately 9 month at approximately 37° C. and approximately pH 7 to 8. Preferred polymer networks of this type are poly(ε-caprolactone) dimethacrylate or urethane dimethacrylate networks that can be copolymerized with e.g. n-butylacrylate, for example poly(ε-caprolactone)-10k urethane dimethacrylate networks or poly(ε-caprolactone)-10k-dimethacrylate/n-butylacrylate networks; poly(ε-caprolactone-co-glycolide) urethane dimethacrylate networks, for example poly(ε-caprolactone-co-glycolide)-10k (97/3) urethane dimethacrylate networks; oligocarbonate-polycaprolactone block copolymer urethane dimethacrylate networks, for example oligocarbonate-polycaprolactone-10k block copolymer urethane dimethacrylate networks; poly(oligocarbonate-sebacate) urethane dimethacrylate network, for example poly(oligocarbonate-sebacate)-8k urethane dimethacrylate networks; and poly(1,6-hexamethylene-adipate)-8k urethane dimethacrylate networks, for example poly(1,6-hexamethylene-adipate)-8k urethane dimethacrylate networks. Abbreviations like-8k or 10k mean, that the respective prepolymer that was crosslinked had a molecular weight of approximately 8,000 g/mol or approximately 10,000 g/mol respectively and the numbers in parenthesis like (97/3) represent weight-%.

Mixed IPN networks that are preferred for the present invention have a shape recovery of at least approximately 85%, and typically from approximately 88 to 94%, have typical switching temperatures from approximately 45 to 55° C. and show no significant biodegradation within approximately 6 month at approximately 37° C. and approximately pH 7 to 8. Such polymers are more flexible and can be processed (e.g., extruded) more easily than the aforementioned pure UV crosslinked dimethacrylate networks and the thermoplasts that are mixed with the prepolymers before crosslinking, can be used to adjust the properties of the mixed IPN networks. Examples of mixed IPN networks that are preferred for the invention are the aforementioned preferred UV crosslinked dimethacrylate networks polymerized in the presence of thermoplasts, in particular in the presence of thermoplastic polyurethanes like Carbothane® or polycaprolactones like CAPA®. Particularly preferred mixed IPN networks of the invention are poly(ε-caprolactone) dimethacrylate, urethane dimethacrylate or urethane tetramethacrylate networks, for example poly(ε-caprolactone)-10k urethane dimethacrylate networks or poly(ε-caprolactone)-16k urethane dimethacrylate or tetramethacrylate networks. Such networks include from approximately 10 to 80 weight-%, preferably from approximately 20 to approximately 70 weight-% and particularly preferred from approximately 25 to approximately 55 weight-% thermoplastic polyurethanes like Carbothane® or polycaprolactones like CAPA®, wherein the thermoplastic polycaprolactones usually have a molecular weight of at least approximately 20,000 g/mol, preferably from approximately 30,000 to approximately 120,000 g/mol and more preferably from approximately 40,000 g/mol to approximately 80,000 g/mol.

For urethane networks no UV crosslinking is necessary, but workpieces usually need to be processed by reaction injection molding. Such networks preferably have a shape recovery of greater than approximately 95%, switching temperatures about approximately 45 to approximately 55° C. and show no significant biodegradation within approximately 15 month at approximately 37° C. and approximately pH 7 to 8. Preferred materials are poly(ε-caprolactone) tetrols or blends of poly(ε-caprolactone) tetrols and diols crosslinked with aliphatic diisocyanates like trimethylhexamethylene diisocyanate (TMDI) or hexamethylene diisocyanate (HMDI), for example poly(e-caprolactone)-16k tetrol/TMDI networks or (poly(ε-caprolactone)-16k tetrol/poly(e-caprolactone)-10k diol) TMDI networks.

Biodegradable materials and in particular biodegradable network materials that can be preferably used for the stent of the invention are, for example, described in U.S. Pat. No. 6,160,084, WO 2004/006885 and WO 2005/028534, that are herewith incorporated in their entirety. Preferred biodegradable shape memory polymers include, for example, from amorphous dimethacrylate or urethane dimethacrylate networks, amorphous urethane networks and amorphous multiblock copolymers.

Biodegradable amorphous dimethacrylate networks typically have a shape recovery of greater than approximately 90%, switching temperatures from approximately 20 to 55° C. and can show greater than approximately 70 weight-% mass loss within approximately 9 month at approximately 37° C. and approximately pH 7 to 8. Preferred examples of such polymers are poly(L-lactide-co-glycolide) dimethacrylate networks or poly(L-lactide-co-glycolide) dimethacrylate/monoacrylate networks, wherein the poly(L-lactide-co-glycolide) dimethacrylate prepolymer preferably has a molecular weight of approximately 3,000 to approximately 10,000 g/mol and in particular from approximately 4,000 to approximately 7,000 g/mol and the monoacrylate includes, for example, n-butylacrylate, n- or cyclo-hexylacrylate, triethylcitrate or caprolactone 2-(methacryloyloxi)ethylester. Some of those polymers are very rigid and brittle and preferably are plasticized by known means.

Biodegradable amorphous urethane networks typically have a shape recovery of greater than approximately 90%, switching temperatures from approximately 40 to approximately 65° C. and a degradation stability of less than approximately 12 month at approximately 37° C. and approximately pH 7 to 8. Preferred materials of this class are based on trihydroxy terminated or tetrahydroxy terminated copolyesters based on rac-dilactide with glycolide, or p-dioxanone or caprolactone and diisocyanates, wherein the diisocyanates preferably are aliphatic compounds like trimethyl hexamethylene diisocyanate or hexamethylene diisocyanate.

Biodegradable amorphous multiblock copolymers typically have switching temperatures from approximately 10 to approximately 40° C. and they usually degrade to more than approximately 50 weight-% in less than one year. Preferred materials are copolymers based on oligo-caprolactone or oligo(lactide-co-glycolide) as soft segment and oligo-p-dioxanone as hard segment. Such copolymers based on oligo-caprolactone and oligo-p-dioxanone typically have shape recovery values of approximately 60 to approximately 70% and those based on oligo(lactide-co-glycolide) and oligo-p-dioxanone of approximately 30 to approximately 60%.

It is also possible to use photosensitive networks to produce the inventive stents. Suitable photosensitive networks are amorphous and feature covalent crosslinking points, which determine the permanent shape of the stent. Another feature is a photoreactive component, or a reversibly light-switchable unit, which determines the temporary shape of the stent.

In the case of the photosensitive polymers, a suitable network is used which includes photosensitive substituents along the amorphous chain segments. On UV irradiation, these groups are capable of entering into covalent bonds with one another. If the material is deformed and irradiated with light of a suitable wavelength λ1, the original network is additionally crosslinked. The crosslinking achieves temporary fixing of the material in the deformed condition (programming). Renewed irradiation with light of another wavelength λ2 can in turn release the crosslinking and thus restore the original shape of the material (regeneration) because the photo-crosslinking is reversible. This type of photochemical cycle can be repeated as often as desired. The basis for the photosensitive materials is a wide-mesh polymer network which, as stated above, is transparent with respect to the radiation intended to trigger the alteration of shape (i.e., preferably forming a UV-transparent matrix). According to the invention, preference is given to networks of the invention based on low-molecular-weight acrylates and methacrylates which can be polymerized by a free-radical route, in particular C1-C6 (meth)acrylates and hydroxy derivatives, preference being given to hydroxyethyl acrylate, hydroxypropyl methacrylate, hydroxypropyl acrylate, poly(ethylene glycol) methacrylate and n-butyl acrylate; n-butyl acrylate and hydroxyethyl methacrylate are preferably used.

The comonomer used to produce the polymeric networks of the invention include a component which is responsible for the crosslinking of the segments. The chemical nature of this component naturally depends on the nature of the monomers.

For the preferred networks based on the acrylate monomers described above as preferred, suitable crosslinking agents are bifunctional acrylate compounds which have suitable reactivity with the starting materials for the chain segments, so that they can be reacted together. These crosslinking agents include short, bifunctional crosslinking agents, such as ethylene diacrylate, low-molecular-weight bi- or polyfunctional crosslinking agents, oligomeric, linear diacrylate crosslinking agents, such as poly(oxyethylene)diacrylates or poly(oxypropylene)diacrylates, and branched oligomers or polymers having acrylate end groups.

The inventive network further includes a photoreactive component (group) which is concomitantly responsible for triggering the controllable alteration of shape. This photoreactive group is a unit which, via excitation by suitable light, preferably UV radiation, can react reversibly (with a second photoreactive group) to generate or separate covalent bonds. Preferred photoreactive groups are those capable of reversible photodimerization. Preferred photoreactive components used in the inventive photosensitive networks are various cinnamates (CA) and cinnamylacylates (GM).

It is known that cinnamic acid and its derivatives dimerize under UV light of about 300 nm, forming a cyclobutane. If the dimers are irradiated with UV light of smaller wavelength, about 240 nm, they can be cleaved again. The absorption maxima can be shifted by substituents on the phenyl ring, but always remain within the UV region. Other derivatives capable of photodimerization are 1,3-diphenyl-2-propene-1-one (chalkone), cinnamylacylic acid, 4-methylcoumarin, various ortho-substituted cinnamic acids, cinnamyloxysilanes (silyl ethers of cinnamyl alcohol).

Photodimerization of cinnamic acid and of similar derivatives is a [2+2]cycloaddition of the double bonds to give a cyclobutane derivative. The E-isomers, and also the Z-isomers, are capable of entering into this reaction. Under irradiation, E/Z-isomerization competes with the cycloaddition reaction. However, E/Z-isomerization is inhibited in the crystalline state. The various possible arrangements of the isomers with respect to one another theoretically permit 11 different stereoisomeric products (truxillic acids, truxinic acids). For the reaction, the required separation of the double bonds of two cinnamic acid groups is about 4 Å.

The networks feature a number of desirable properties. Overall, the networks are good SMP materials with high recovery values, meaning that a high percentage, usually above 90%, of the original shape is regained even on repeated passage through a cycle of changes of shape. Nor does any disadvantageous loss of mechanical properties occur here.

Additionally, the SMP materials used are hydrolysable or biodegradable because the abovementioned materials are based on aliphatic polyesters. Surprisingly, it has been found that on the one hand these materials decompose in a biocompatible manner (i.e., giving non-toxic degradation products) while on the other hand the mechanical integrity of the stent is retained during the degradation process for a long time, ensuring sufficiently long functionality of the stent.

In one preferred embodiment of the inventive tubular tissue supports, the folding takes place inwards in the longitudinal axis of the tube in the temporary shape.

In another preferred embodiment, the tube can be folded inwards two or more times. By way of example, there can be from approximately 2 to approximately 16 folds.

The tube length of the stents is generally in the range from approximately 1 to approximately 15 cm, their diameter being in the range from approximately 1 to approximately 15 mm and their thickness from approximately 50 to approximately 1,000 μm, preferably from approximately 75 to approximately 500 μm and more preferably from approximately 100 to approximately 400 μm.

The stents of the invention are preferably biocompatible (class III according to DIN/ISO 10993). Namely, the stents of the invention are non-cytotoxic, hemocompatible and non-inflammatory, withstand radial forces of at least approximately 0.4 bar, and the non-biodegradable stents have a degradation stability of at least approximately 6 month without significant mass loss at approximately 37° C. and approximately pH 7 to 8.

Additionally, if it is intended to remove the stent after a given time, there should be poor cell in-growth and adhesion of cells to the material. On the other hand, if the biodegradable stent is intended to resolve in the body, it should have good cell in-growth and adhesion of cells to the material to exert high radial forces as long as possible. Regardless, both types of stent should not cause an occlusion. The shape of the tube of the inventive tissue supports corresponds to the shape of the tissue requiring support. Accordingly, they can have a straight or curved shape.

The invention also includes a process for producing radially expandable tubular tissue supports in the temporary shape of a shape-memory material that includes: (1) the tube with the shaping of its permanent shape is converted to its temporary shape by heating at or above the transition temperature Tₜᵣₐₙₛ of the shape-memory material; (2) the tube is folded one or more times within itself via pressure of one or more segments into the longitudinal axis of the tube; and (3) the folded tube is stabilized in its temporary shape by reducing the temperature below Tₜᵣₐₙₛ.

The transition temperature of the shape-memory material for the inventive stents is generally in the range from approximately 20 to approximately 70° C., preferably in the range from approximately 30 to approximately 50° C., and more preferably in the range from approximately 35 to approximately 45° C. It is possible to embed heating elements in SMP material in order to set the transition temperature of the inventive stents. However, it is preferable that no heating element is embedded into the SMP material.

As an alternative, the shape-memory effect can also be triggered by using IR radiation, NIR radiation, by applying an oscillating electrical field and/or by UV irradiation.

The invention, therefore, also includes a process for producing radially expandable tubular tissue supports in the temporary shape of a shape-memory material that includes: (1) the tube with the shaping of its permanent shape is converted to its temporary shape by irradiation; (2) the tube is folded one or more times within itself via pressure of one or more segments into the longitudinal axis of the tube; and (3) the folded tube is stabilized in its temporary shape by reducing the temperature below Tₜᵣₐₙₛ.

Examples of radiation that can be used, for example, include IR radiation, NIR radiation, by applying an oscillating electrical field and/or by UV irradiation.

An example of IR radiation is electromagnetic radiation in the range from approximately 2.5 to approximately 25 μm, preferably in the range from approximately 4.0 to approximately 7.0 μm. The radiation source can, by way of example, be introduced in the form of a probe into the stent.

An example of NIR (near infrared) radiation is electromagnetic radiation in the range from approximately 700 to approximately 2,500 nm, preferably in the range from approximately 800 to approximately 1,500 nm.

An example of UV radiation is electromagnetic radiation in the range from approximately 200 to approximately 500 nm, preferably in the range from approximately 250 to approximately 350 nm.

The diameter of a segment which is pressed into the longitudinal axis of the tube for folding of the tube in its temporary shape is preferably smaller than the diameter of the tube. A segment is generally round (rod). It can, however, be oval or assume angular shapes. The diameter of the rod is particularly preferred to be smaller than the diameter of the tube by from approximately 10 to approximately 50%. The shape of the rod for folding of the tube for the purposes of the invention is preferably that of the tube in its permanent shape.

In one preferred embodiment of the invention, the folded tube in the temporary shape can also be compressed by repeated rolling. In another preferred embodiment, it is also possible to press two or more rods into the tube, as described, for example, in WO 2004/010901 A1.

In another preferred alternative of the inventive process it is possible to compress the stent in the temporary shape into individual segments. Preferably this processing is done with a compressing or crimping tool and/or a method such as that described in U.S. Pat. No. 6,629,350 (the disclosure of which is expressly incorporated herein by reference in its entirety). By crimping the stent, a round cross section with a regular, and when using many segments, almost flat (smooth) surface can be achieved, that facilitates the deployment. The expansion to the permanent form proceeds in a very reproducible manner by triggering the shape memory effect and the wall of the resulting implanted tube has uniform thickness and exerts high radial forces. In a preferred embodiment of the invention, the temporary form of the stent is achieved by directly crimping it onto the balloon of the catheter, that is used for the deployment.

By way of example, the inventive stents can be shaped (i.e., “programmed”) as follows: (1) the stent gains its permanent shape in a manner known per se, for example, via injection moulding or extrusion; (2) the stent, in its permanent shape, is heated to a temperature greater than Tₜᵣₐₙₛ, to produce the temporary shape; (3) during the programming process to its temporary shape, the inventive stent is converted to a diameter which is smaller than the original diameter. According to the invention, this sizing of the diameter can take place via pressing of a rod into the longitudinal axis of the tube; the tube is thus folded within itself, (4) the folded stent is then cooled to a temperature lower than Tₜᵣₐₙₛ to fix the temporary shape of the stent; and (5) the stent, while cooled to a temperature lower than Tₜᵣₐₙₛ, is drawn out of the production process with the aid of a guide wire or of a guide thread and can be assembled onto a suitable catheter.

The invention also provides the use of folded tissue supports composed of shape-memory material in the temporary shape for introduction into a relevant vessel.

Minimally invasive insertion of a stent into a hollow organ cans by way of example, be described as follows: (1) the stent, provided on a temperature-controllable balloon catheter, is introduced into the tubular organ by the minimally invasive method; (2) the fitted stent is heated by means of a catheter above its Tₜᵣₐₙₛ (balloon fills with warm water (liquid) or gas), or is irradiated with light of wavelength smaller than 260 nm. The stent expands and widens during this process; and (3) the stent now has its permanent shape (expanded) and the balloon catheter can be removed.

What is claimed is:

1. A radially expandable tubular tissue support comprising at least one shape-memory material, wherein said radially expandable tubular tissue support comprises a tube in the temporary shape of the shape-memory materials, and wherein said tube has been folded one or more times along its longitudinal axis.

2. The tubular tissue support according to claim 1, wherein the tube is folded inwards.

3. The tubular tissue support according to claim 1, wherein the length of the tube is in the range from approximately 1 to approximately 15 cm and its diameter is from approximately 1 to approximately 15 mm.

4. The tubular tissue support according to claim 1, wherein a permanent shape of the tubular support has one of a straight shape or a curved shape.

5. The tubular tissue support according to claim 1, wherein the shape-memory material comprises at least one polymer.

6. The tubular tissue support according to claim 1, wherein the polymer is at least one of a networks, a thermoplastic or a blend.

7. (canceled)

8. A process for production of a radially expandable tubular tissue support in a temporary shape of a shape-memory material, wherein a tube with a shaping of its permanent shape is converted to its temporary shape via heating at or above a transition temperature Tₜᵣₐₙₛ of the shape-memory material, comprising the steps of: folding the tube one or more times within itself via pressure of one or more segments into the longitudinal axis of the tube and stabilizing the folded tube in its temporary shape by reducing the temperature below Tₜᵣₐₙₛ.

9. The process according to claim 8, wherein the transition temperature of the shape-memory material is in the range from approximately 20° C. to approximately 70° C.

10. The process according to claim 9, wherein the transition temperature of the shape-memory material is in the range from approximately 30° C. to approximately 50° C.

11. The process according to claim 10, wherein the transition temperature of the shape-memory material is in the range from approximately 35° C. to approximately 45° C.

12. A process for production of a radially expandable tubular tissue supporting a temporary shape of a shape-memory material, comprising the steps of: converting a tube with the shaping of its permanent shape into a temporary shape by irradiation; folding the tube one or more times within itself via pressure of one or more segments into the longitudinal axis of the tube; and stabilizing the folded tube in its temporary shape by reducing the temperature below Tₜᵣₐₙₛ.

13. The process according to claim 12, wherein a diameter of a segment is smaller than the diameter of the tube.

14. The process according to claim 13, wherein a diameter of a segment is from approximately 10% to approximately 50% smaller than the diameter of the tube.

15. The process according to claim 14, wherein the shape of a segment is that of the tube in its permanent shape.

16. The process according to any of claim 15, wherein two or more segments are pressed from outside into the tube.

17. The use of folded tissue supports composed of a shape-memory material in a temporary shape for introduction into a relevant vessel.
